SEQUENCE LISTING (2 sequences)

SEQ ID NO: 1 - protein, 486 aa, Unknown
  Found in ikaite columns of SW Greenland
MDNGLMFQGFEWYMPDDGNYYKDLKKKLVDMKRIGVTSVWLPPVCKATGSNDTGYGVYDLYDLGEFDQKGSVRTKYGTKEELLDLIKAIHDEGMYVYADVVLNHKAGADFEEEFMAVKVDNNNRTKEIEKQRNIKAWTGFNFPGRNGKYSDFTWNYNHFSGVDYDASTGDKGIFRIIGENKGWNWGVSHDNGNFDYLMFADIDHANTEVKEELKRWVDWFIEELNLDGIRFDAVKHIDSAFLEEFTSHIKEKMGDEFYFLGEYWDHDVKNKIKFMKSTKYSMDLFDVGLHFNMYAASQNSANYDLRKLFDNTVTKTDPAMSVTFVDNHDSEPGQSLESFVKEWFKEIAYGIILLRKDGYPCIFYGDYYGIGGEFMIKPLKEKIDVLSLIRKNHAYGAQDDYFKEKDLIGWVRQGTEDHPGKCAVVISTREKKTISMFIDKYHSGKVYADFTGNCADKVKVDEEGYGEFTAEAGSISVWLEEEIVLG

SEQ ID NO: 2 - DNA, 1461 bp, Unknown
  Found in ikaite columns of SW Greenland
atggacaatggattaatgtttcagggattcgaatggtatatgcctgatgacgggaattattataaagatcttaagaagaagctggtagacatgaagcgcataggcgtcacatcagtctggctgccaccggtatgcaaggcaacagggtcaaatgatacgggatatggagtctatgatctctatgatcttggggagtttgatcagaaaggttccgtgcggacaaagtatggtacaaaggaagaacttcttgatctgatcaaagccatccatgatgaaggaatgtatgtctacgcggatgtggttctgaatcacaaagcgggagcagatttcgaggaagagttcatggcggtgaaagtggataacaataaccgcacgaaggaaattgaaaagcaaagaaatatcaaagcatggacaggattcaactttccaggaagaaatggaaagtattctgatttcacatggaactacaatcatttttccggtgtggattatgatgccagtacaggagataagggtattttcagaatcatcggagaaaataaggggtggaactggggcgtatcccatgacaacggaaacttcgattatctgatgtttgcggacattgaccatgcgaatacggaagtgaaggaagaattaaagagatgggtggactggttcattgaagagctaaacctggacggaatccgttttgatgcagtgaaacatattgacagtgcgttccttgaagagttcacaagccatatcaaagaaaagatgggtgatgaattctattttctgggagagtactgggatcatgacgtgaaaaataaaataaaattcatgaaatccaccaaatacagtatggatctttttgatgtcggtcttcatttcaacatgtatgccgcatcccagaattcggccaactacgatcttcggaaactgtttgacaataccgtaactaaaaccgatccagccatgagcgtcacttttgtggacaatcatgattcagaaccgggacaatctctggagtcctttgtcaaggaatggttcaaggaaatcgcctatggtatcatccttcttcgtaaagatggatatccctgtattttttatggggattattacggcatcggcggagagtttatgatcaaaccactgaaagagaagatcgatgttctttcgctgatcagaaagaatcatgcttacggagctcaggacgactacttcaaggaaaaagatctcatcggctgggtaagacagggaacggaagatcatccaggaaaatgtgcagtggtgatttcgaccagggagaagaagactatttccatgttcatagacaaatatcattctggaaaagtatatgcggatttcactggtaactgtgcagataaggtgaaggtggacgaagagggctatggagagttcactgctgaagcaggcagtatttccgtatggcttgaagaagaaattgtgcttggctga

(19) United States
(12) Patent Application Publication
(10) Pub. No.: US 2017/0044510 A1
(43) Pub. Date: Feb. 16, 2017

(54) COLD-ACTIVE ALPHA-AMYLASE

(71) Applicant: COLDZYMES APS, Frederiksberg C (DK)

(72) Inventors: Peter STOUGAARD, Kirke Hyllinge (DK); Jan Kjoelhede VESTER, Frederiksberg C (DK); Mikkel Andreas GLARING, Frederiksberg C (DK)

(21) Appl. No.: 15/307,509

(22) PCT Filed: Apr. 28, 2015
(86) PCT No.: PCT/DK2015/050108

§ 371 Date: Oct. 28, 2016

(30) Foreign Application Priority Data
Apr. 29, 2014 (DK) PA 2014 70249

(51) Int. Cl. C12N 9/28

(52) U.S. Cl. CPC C12N 9/2417; C12Y 302/01001

(57) ABSTRACT

There is provided a novel cold-active alpha-amylase identified by a functional metagenomic approach expressed in E. coli and purified to homogeneity. Functional, biochemical analysis has documented that the alpha-amylase is cold-adapted with a temperature optimum at 10° C. to 20° C. and that the enzyme is active over a broad pH range. Sequence analysis has indicated that the alpha-amylase is related to Clostridia, and has revealed classical characteristics of cold-adapted enzymes.

### FIELD OF THE INVENTION

The present invention relates to a novel cold-active alpha-amylase and DNA encoding the enzyme.

### BACKGROUND OF THE INVENTION

Alpha-amylases (alpha-1,4-glucan-4-glucanohydrolases, E.C.3.2.1.1) constitute a group of enzymes, which catalyze the hydrolysis of starch and other linear and branched 1,4-glucosidic oligo- and polysaccharides.

Alpha-amylases are used commercially for a variety of purposes such as in the initial stages of starch processing (e.g., liquefaction); in wet milling processes; and in alcohol production from carbohydrate sources. They are also used as cleaning agents or adjuncts in detergent matrices; in the textile industry for starch desizing; in baking applications; in the beverage industry; in oil fields in drilling processes; in recycling processes, e.g., for de-inking paper; and in animal feed.

One of the first bacterial alpha-amylases to be used was an alpha-amylase from B. licheniformis, also known as Termamyl, which has been extensively characterized and the crystal structure has been determined for this enzyme. Alkaline amylases, such as the alpha-amylase derived from Bacillus sp. strains NCIB 12289, NCIB 12512, NCIB 12513, and DSM 9375 (disclosed in WO 95/26397), form a particular group of alpha-amylases that are useful in detergents. Many of these known bacterial amylases have been modified in order to improve their functionality in a particular application.

Termamyl and many highly efficient alpha-amylases require calcium for activity. The crystal structure of Termamyl shows that three calcium atoms are bound to the alpha-amylase structure coordinated by negatively charged amino acid residues. This requirement for calcium is a disadvantage in applications where strong chelating compounds are present, such as in detergents or during ethanol production from whole grains, where the plant material comprises a large amount of natural chelators such as phytate.

While thermostable amylases are needed for starch liquefaction, the use of cold-active amylases can be highly beneficial for other applications. In the baking industry, amylases are used to improve bread softness and volume as well as to prevent stalling (Kirk et al. 2002; Gupta et al. 2003; Bisgaard-Frantzen et al. 1999), after which complete inactivation of the enzyme is required (Coronado et al. 2000). This can be accomplished using heat labile cold-active enzymes. The use of cold-active enzymes is especially promising in laundry and dish-washing detergents, where they allow for environment-friendly low temperature washing (Mojallali et al. 2013; van der Maarel et al. 2002). Besides being cold-active, enzymes used in detergents also have to be alkali-tolerant (Gupta et al. 2002).

Very few cold-active alpha-amylases have been reported. The alpha-amylase from Clostridium perfringens retains 70% of its activity at 15° C. (Shih and Labbe 1995), enzymes from natural isolates related to Actinobacteria (Groudieva et al. 2004) and Bacilli (Mojallali et al. 2013) retained 20% and 13% of their activity at 0° C., respectively, and the alpha-amylase from a Bacillus cereus strain retained 50% of the activity at 10° C. (Mandavi et al. 2010). All these cold-active alpha-amylases have pH optimum at pH 6-7. The alpha-amylase from Pseudoalteromonas arctica GS230 (Lu et al. 2010) and from an Actinomycete strain related to Nocardiopsis (Zhang and Zeng 2008) showed a somewhat broader pH optimum (7-8.5 and 7-9, respectively) and retained 34.5% and ˜25% of the activity at 0° C., respectively.

Therefore, in order to develop a low-temperature process for hydrolysis of starch there is a need for a novel cold-active alpha-amylase.

### SUMMARY OF THE INVENTION

The present invention provides a purified cold-active alpha-amylase. Specifically, the present invention provides a cold-active alpha-amylase having the sequence as defined in SEQ ID NO 1, or one having at least 80% homology (or sequence identity) to the amino acid sequence as defined in SEQ ID NO. 1,wherein the amino acid sequence preferably being selected so that the enzyme has a stable enzymatic activity at temperatures less than 10° C. Preferably the amino acid sequence has at least 90%, and more preferably 95%, homology (or sequence identity) to the amino acid sequence as defined in SEQ ID NO. 1.

In a further embodiment, the present invention provides a recombinant vector comprising a DNA sequence that encodes a protein with an amino acid sequence as given in SEQ ID NO 1 or one having at least 80% homology (or sequence identity) to the amino acid sequence as defined in SEQ ID NO. 1.

Another object of the present invention is a strain of an isolated bacterium capable of producing a cold-active alpha-amylase according to the present invention.

Another object of the invention is a recombinant plasmid or vector suited for transformation of a host, capable of directing the expression of a DNA sequence according to the invention in such a manner that the host expresses the cold-active α-amylase of the present invention in recoverable form.

According to the invention, another object is the so transformed host. A variety of host-expression systems may be conceived to express the cold-active alpha-amylase coding sequence, for example bacteria, yeast, insect cells, plant cells, mammalian cells, etc. Particularly, in yeast and in bacteria, a number of vectors containing constitutive or inducible promoters may be used.

It is also an object of the present invention to provide a process for purifying the cold-active alpha-amylase according to the present invention from a bacterium as well as to provide a process for producing cold-active alpha-amylase according to the invention in a transformed host.

Accordingly, the invention pertains to a method of producing a polypeptide having cold-active alpha-amylase activity, comprising isolating a DNA fragment encoding the polypeptide, inserting said DNA fragment into an appropriate host organism, cultivating the host organism under conditions, which lead to expression of the a polypeptide with cold-active alpha-amylase activity and recovering said polypeptide from the cultivation medium or the host organism.

An appropriate host organism is preferably selected from the group consisting of Escherichia, Bacillus, Bifidobacterium, Lactococcus, Lactobacillus, Streptomyces, Leuconostoc, Streptomyces, Saccharomyces, Kluyveromyces, Candida, Torula, Torulopsis, Pichia and Aspergillus.

In a further aspect, the invention relates to a recombinant DNA molecule comprising a DNA fragment encoding a polypeptide having cold-active alpha-amylase activity and to a microbial cell comprising such recombinant DNA molecule.

### DESCRIPTION OF THE DRAWINGS

FIG. 1 shows the phylogenetic affiliation of the present alpha-amylase (Amy_{I3C6}) compared to close relatives in the GenBank Reference Proteins database. Percent identity to Amy_{I3C6} is noted for each protein as well as the accession number. Numbers on branches are bootstrap values. Taxonomy at class level is presented to the right. A: Archaea, γ-Prot.: γ-Proteobacteria.

FIG. 2 shows (A) SDS-PAGE and (B) native amylopectin-containing gel of purified Amy_{I3C6} (indicated by the arrow). Lane 1 and 5: Molecular weight markers; lane 2: Crude extract; lane 3: Pool after affinity-purification; lane 4: Pool after anion-exchange purification; P: Protein stained with Coomassie Brilliant Blue G-250; 0, 30, 60: Minutes incubated in buffer before staining with Lugol iodine solution.

FIG. 3 shows temperature (A) and pH (B) profiles of crude extracts and purified Amy_{I3C6}. Profiles for the commercially available, low-temperature alpha-amylase Stainzyme® are also included. The residual activity after 60 min at the given temperature (C) and 14 hours at the given pH (D) is also presented. Error bars show standard deviation.

FIG. 4 shows effect of surfactants and inhibitors on Amy_{I3C6} activity.

FIG. 5 shows comparison of temperature profiles of activity of cold-adapted alpha-amylases. A: Amy_{I3C6}, B: Aeromonas veronii NS07, C: Pseudoalteromonas arctica GS230, and D: Nocardiopsis sp. 7326.

### DETAILED DESCRIPTION OF THE INVENTION

As described below the alpha-amylase of the present invention has been expressed recombinantly in E. coli, and pH- and temperature-profiles have been determined as well as analyses of functionality in the presence of detergents and inhibitors. pH optimum is from 8 to 9 but the enzyme is also stable and active in a much larger pH area from pH 6 to pH 10. The temperature optimum is determined to be at 10° C., with more than 60% activity at 1° C. The alpha-amylase is stable at temperatures from minus 20° C. to 28° C. and can be irreversibly inactivated at temperatures above 28° C. The alpha-amylase of the present invention is dependent on calcium-ions, and activity is generally increased in the presence of detergents. In the following the enzyme of the present invention is referred to as Amy_{I3C6}.

### Sequence Based Analysis

All alpha-amylase sequences were downloaded from GenBank (http://www.ncbi.nlm.nih.gov/protein/): Pseudoalteromonas haloplanctis (AHA), GI: 2879820; Bacillus cereus (BCA), GI: 166237002; and Escherichia coil (AmyA), GI: 146023. The closest related sequences to Amy_{I3C6}, the putative alpha-amylases from Finegoldia magna (GI: 488924411) and Helcococcus kunzii (GI: 491541048), were identified using BLASTp with the sequence of Amy_{I3C6} as query against non-redundant protein sequences. Protein sequences used for the phylogenetic analysis were obtained by BLASTp with Amy_{I3C6} as query against the GenBank Reference Proteins (refseq_protein) database. Alignments were constructed in CLC Main Workbench version 6.9 (http://www.clcbio.com/) using default parameters. Phylogenetic trees were produced in MEGA6 by Neighbor-joining with p-distance and a bootstrapping value of 1,000 (Tamura et al. 2013). Calculations of protein properties were made using the ExPASy ProtParam tool (http://web.expasy.org/protparam/).

### Expression and Purification

The Amy_{I3C6} gene sequence, amy_{I3C6}, was obtained by direct end-sequencing of the purified IKA3C6 BAC library clone, and the DNA sequence was used to identify the contig harbouring amy_{I3C6} in the corresponding metagenomic sequence of the library (Vester et al. 2014). The sequence encoding amy_{I3C6} was PCR amplified with the primers amy_{I3C6}-F: ATATCATATGGACAATGGATTAATG and amy_{I3C6}-R: ATATCTCGAGGCCAAGCACAATTTC (NdeI and XhoI sites underlined, respectively). The PCR product was digested with NdeI and XhoI and cloned into the expression vector pET21b with a C-terminal 6×His-tag and transformed into E. coil Tuner cells (Novagen). Clones producing alpha-amylase were identified by hydrolysis of AZCL-amylose on LB plates supplemented with 100 μg/mL ampicillin, 1 mM IPTG and 0.05% (w/v) AZCL-amylose (Megazyme). Expression was performed in LB medium supplemented with 100 μg/mL ampicillin by inoculating a culture to an OD₆₀₀ of 0.2, incubating at 37° C. to an OD₆₀₀ of 0.8, then inducing expression by adding 1 mM IPTG and incubating for 16 hours at 20° C. Cell pellets were harvested after 22 h and resuspended in 2 mL Binding Buffer (20 mM sodium phosphate, 500 mM sodium chloride, 20 mM imidazole, pH 7.4). Intracellular proteins were extracted by bead beating in a FastPrep (Thermo Scientific) with 3 cycles of 25 sec at a setting of 5.5 with cooling on ice between cycles. The supernatant was recovered by centrifugation at 10,000 g for 5 min at 4° C. and the enzyme was purified on a Biologic LP system (Bio-Rad) using a 5 mL HisTrap FF column (GE Healthcare) by eluting in 2 mL fractions at a flow rate of 2 mL/min with a 40 mL linear gradient of 0-500 mM imidazole. Fractions containing alpha-amylase activity, determined by AZCL-amylose hydrolysis, were pooled and buffer was changed to 20 mM Tris-HCl pH 7.6 by ultracentrifugation in a 30 kDa cut-off Vivaspin 20 column (GE Healthcare). Amy_{I3C6} was further purified by subsequent anion exchange using a 1 mL HiTrap Q FF column (GE Healthcare) with a final sodium chloride concentration of 1 M. Active fractions were analyzed by SDS-PAGE to determine purity and then pooled. Protein concentration was determined with the BCA Protein Assay Kit (Pierce). Confirmation of correspondence between the observed band on the SDS-PAGE and activity was carried out in an in-gel enzyme assay with 0.2% (w/v) amylopectin. The gel was run on ice at 100V for approximately 3 h and subsequently incubated in 100 mM Tris-HCl buffer, pH 8.3 with 10 mM calcium carbonate for 0, 30 or 60 min and stained with either Coomassie Brilliant Blue G-250 or Lugol iodine solution (Sigma-Aldrich) to visualize enzyme activity.

### Temperature, pH and Stability Assays

Temperature and pH profiles of activity was determined in crude extract and on purified Amy_{I3C6} using an assay for reducing-end sugars as described by Anthon and Barrett (2002). Assays were performed in 100 mM buffer (Tris-HCl adjusted to pH 8.6 at individual temperatures in the temperature profile experiment and for the pH profile experiment Tris-HCl buffer pH 6-9, glycine-NaOH buffer pH 8-10) with incubation for 10 min and 5 mg/mL amylopectin as substrate. The pH profile was assayed at 20° C. and no buffer effect was observed on activity. Assays with Stainzyme® (Novozymes A/S) were conducted under the same conditions. Stability tests were performed by incubating 5 μL of purified enzyme at the given temperature and time, then keeping the mixture on ice before performing the reducing-ends assay at 20° C. with 5 mg/mL amylopectin in 100 mM Tris-HCl, pH 8.5 with 10 mM calcium carbonate. All analyses were performed in triplicates.

### Effect of Ions, Surfactants, Inhibitors and Detergents

The effect of ions, surfactants, inhibitors, and detergents was determined in assays containing 5 mg/mL amylopectin as substrate in 100 mM Tris-HCl, pH 8.6 and incubated at 20° C. for 10 min. Activity was measured as a release of reducing sugars from amylopectin as described by Anthon and Barrett (2002). For detergents, tap water was used in addition to buffer and the pH was adjusted to 8.6 using HCl. For solid detergents, 5 mg/mL was used and liquid detergents were diluted 100-fold to simulate washing conditions. Two of the detergents used contained added amylases: Grøn Balance White Wash and Bio-tex, whereas the third, Neutral General Purpose did not. Assays were performed in a 100 μL total volume with 5 μL purified Amy_{I3C6}. All analyses were performed in triplicates, and amylase activity from detergents was subtracted from the results.

### Substrate Specificity

Hydrolysis of amylopectin from potato (Fluka), amylose (Hayashibara Biochemical Laboratories), granular starch (Merck), and glycogen (Sigma) was conducted as a time series at 15° C. in 600 μL reactions containing 100 mM Tris-HCl, pH 8.5, 10 mg/mL of the substrate and 5 μL purified enzyme. Assays were stopped by adding 100 μL 1M NaOH to extracted subsamples of 100 μL after 1, 2, 3, 4, and 16 h. Hydrolyzed starch from potato (Sigma) and maltooligosaccharides (G2-G5+G7) were hydrolyzed in an identical assay and stopped after 72 h. Digestion products were analyzed on TLC aluminium sheets (Merck) running in a 1-butanol:2-propanol:water (3:12:4) mixture.

### RESULTS

### Sequence Based Analyses

The amino acid sequence of Amy_{I3C6} was compared to the sequences of alpha-amylases from the psychrophilic Pseudoalteromonas haloplanctis (AHA), the broad temperature-range Bacillus cereus (BCA), and the mesophilic Escherichia coli (AmyA) (Table 1).

TABLE 1
Comparison of Amy_{I3C6} to alpha-amylases from the psychrophilic P. haloplanctis
(AHA), the broad temperature-range B. cereus (BCA), and the
mesophilic E. coli (AmyA).

| | | AHA* | BCA* | AmyA |
|---|---|---|---|---|
| Parameter | Amy_{I3C6} | (psychrophilic) | (broad) | (mesophilic) |
| Calculated size | 486aa | 453aa | 486aa | 495aa |
| | 56.07 kDa | 49.34 kDa | 55.31 kDa | 56.64 kDa |
| Closest relatives | Finegoldia magna | — | — | — |
| | 58% identity | 12% identity | 43% identity | 38% identity |
| | GI: 488924411 | GI: 2879820 | GI: 166237002 | GI: 146023 |
| Arginine | 2.7% | 2.9% | 3.3% | 4.0% |
| Arginine/(Arginine + Lysine) | 0.22 | 0.50 | 0.36 | 0.50 |
| ratio | | | | |
| Proline | 1.9% | 2.9% | 3.5% | 4.6% |
| Active site | YFLGEYWDHD | VGASEYLSTGL | FTVAEYWQND | FIVAEYWSHE |

*For BCA and AHA, signal sequences were identified with SignalP (Petersen et al. 2011) and removed before analysis.

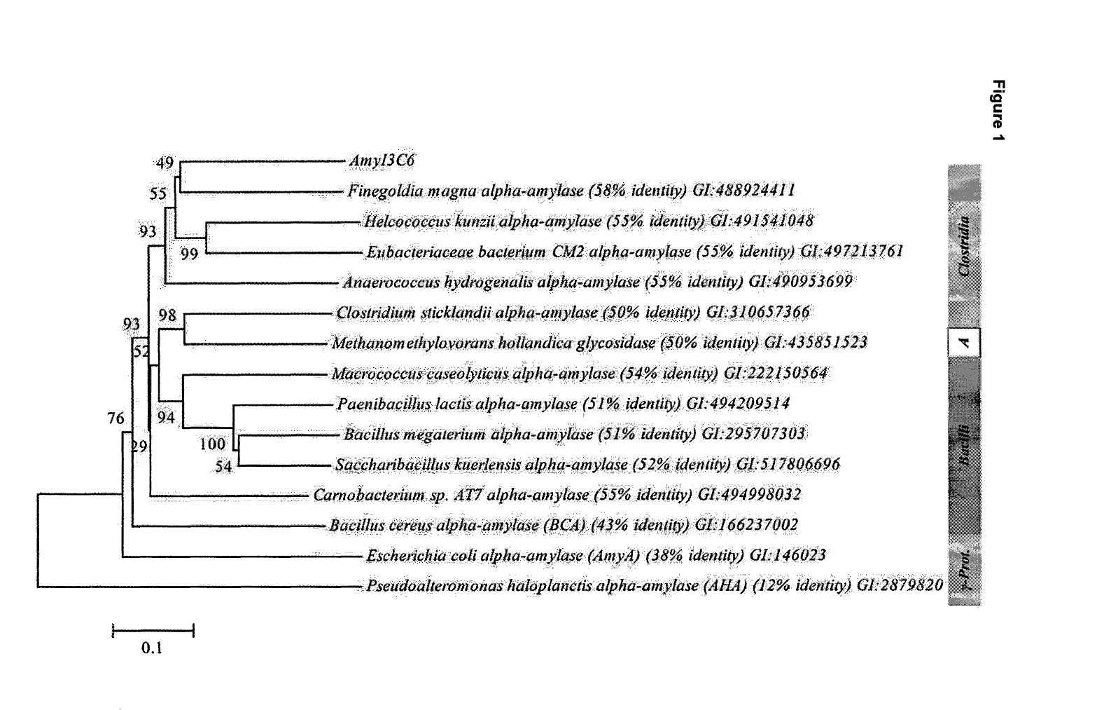

All four alpha-amylases were of similar size and Amy_{I3C6} showed a more pronounced adaption to low temperature than AHA when compared to AmyA (lower arginine, proline and arginine/(arginine+lysine) ratio). The proton donor of the active site glutamic acid (E) and the neighboring tyrosine (Y) were conserved in all four proteins. The closest relative to Amy_{I3C6} was a putative alpha-amylase from Finegoldia magna (58% identity) within the class Clostridia. A phylogenetic analysis of Amy_{I3C6} and the closest relatives in the GenBank Reference Proteins database as well as AHA, BCA and AmyA, showed that Amy_{I3C6} clustered with alpha-amylases of the class Clostridia (FIG. 1).

The 25 amino acids constituting the catalytic cleft and involved in substrate binding are all strictly conserved between the psychrophilic AHA and the mesophilic pig alpha-amylase (Cipolla et al. 2011) and an alignment of Amy_{I3C6} and other relevant alpha-amylases revealed that 16 of these were also conserved in Amy_{I3C6} while two were changed conservatively. Six amino acids were conserved across kingdoms between a thermophilic (Bacillus), a mesophilic (Barley) and hyperthermophilic (Archea) alpha-amylase (Linden and Wilmanns 2004), and all six sites were also present in Amy_{I3C6}.

### Production and Purification of Amy_{I3C6}

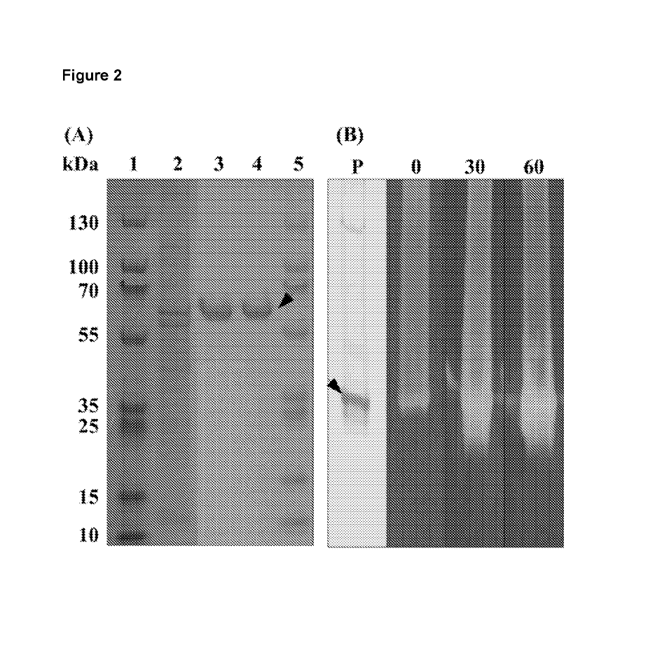

Amy_{I3C6} was produced recombinantly in E. coli with a C-terminal polyhistidine-tag and purified to apparent homogeneity in a two-step process involving affinity-purification and subsequent anion exchange. The purity of the final preparation was evaluated by SDS-PAGE and alpha-amylase activity was confirmed by separation on a native amylopectin-containing gel (FIG. 2).

### Temperature and pH Optimum and Stability Profile of Amy_{I3C6}

### Temperature and pH Optimum

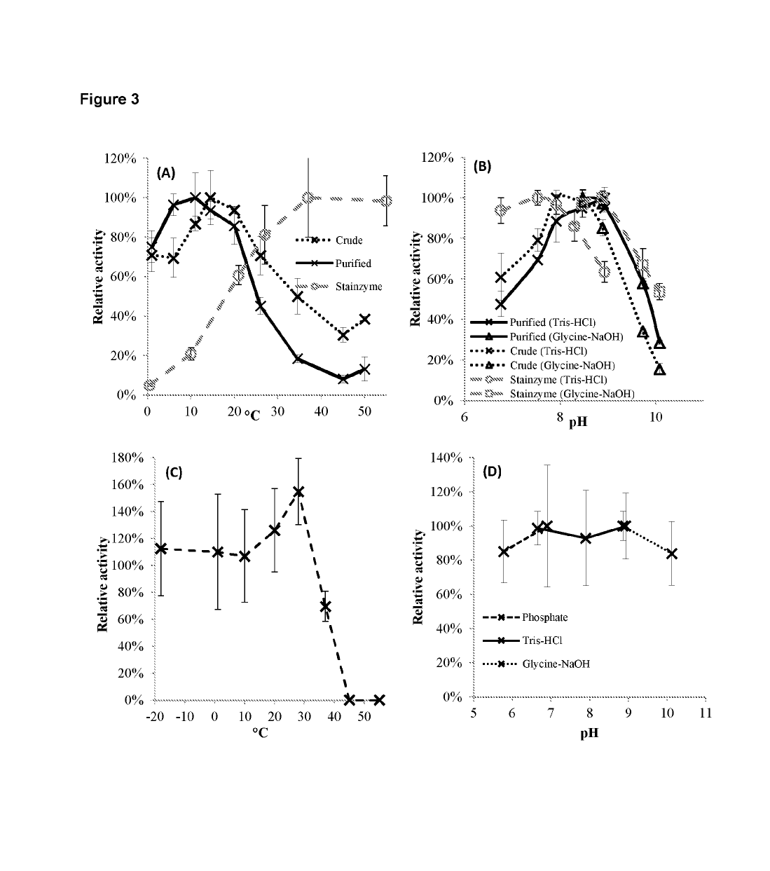

Temperature and pH profiles were determined by an assay for reducing-end sugars after 10 min incubation with amylopectin as substrate. The temperature profile of Amy_{I3C6} showed an optimum at 10-15° C. with more than 70% of the activity retained at 1° C. (FIG. 3). The pH optimum was at pH 8-9, and the enzyme was active at both pH 6.8 and 10. The corresponding profiles of the commercial low-temperature alpha-amylase Stainzyme® (Novozymes, A/S) was obtained for comparison. Stainzyme® had a temperature optimum at 37° C. and 20% activity at 10° C., with pH optimum from 7 to 9.

### Heat-Lability and pH Stability

Sensitivity to heat and pH stability of Amy_{I3C6} was determined by pre-incubating the enzyme at the given temperature or pH followed by an assay for reducing-end sugars after 10 min incubation with amylopectin as substrate at 20° C. Temperature lability tests of Amy_{I3C6} showed no appreciable loss of activity during 60 min incubation at 28° C. or below (FIG. 3), whereas activity was completely lost after 5 min at 55° C., 20 min at 45° C., or three hours at 37° C. (data not shown) illustrating that Amy_{I3C6} is indeed a heat-labile enzyme that can easily be irreversibly inactivated. It was, however, stable for at least 13 days at 1° C. (data not shown). The enzyme was stable in the range of pH 6-10 for at least 14 hours when assayed at 20° C. (FIG. 3).

### Effect of Ions, Inhibitors, Surfactants and Detergents on Amy_{I3C6}

### Ions

The effect of various metal-ions as well as carbonate-ions on the activity of Amy_{I3C6} was determined by addition of 2 mM of the relevant ion to assays on amylopectin at 20° C. (Table 2). Calcium-, barium- and magnesium-chloride led to a slight decrease in activity. Iron(II)chloride had a moderate negative effect, whereas zinc- and copper(II)chloride showed strong negative effects on activity. No significant effect was observed for carbonate ions.

TABLE 2
Effect of ions on the activity of Amy_{I3C6}. Superscript letters denote
groups that are statistically different from the control experiment
(p < 0.001). Standard deviations are given in parentheses.

| Ions | Relative activity | |
|---|---|---|
| H₂O | 100% | (±1.2%) |
| Ca²⁺ (CaCl₂) | 86% | (±0.8%)^{A} |
| Ba²⁺ (BaCl₂) | 81% | (±1.2%)^{A} |
| Mg²⁺ (MgCl₂) | 80% | (±3.6%)^{A} |
| Fe²⁺ (FeCl₂) | 55% | (±2.8%)^{B} |
| Cu²⁺ (CuCl₂) | 8.1% | (±0.3%)^{C} |
| Zn²⁺ (ZnCl₂) | −0.1% | (±0.1%)^{C} |
| CO₃²⁻ (CaCO₃) | 107% | (±1.2%) |
| CO₃²⁻ (NH₄CO₃) | 95% | (±1.1%) |

### Surfactants and Inhibitors

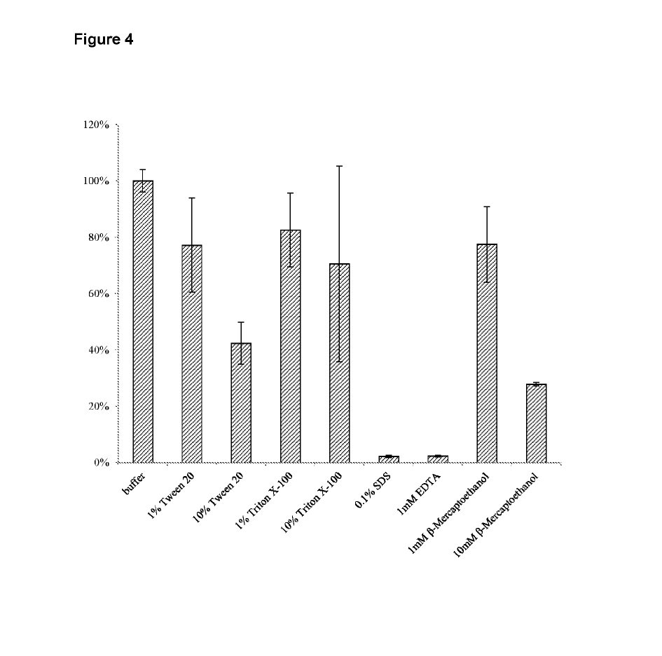

The effect of surfactants and inhibitors on the activity of Amy_{I3C6} was tested by direct addition of the compounds to assays on amylopectin at 20° C. (FIG. 4). The non-ionic surfactants Tween 20 and Triton X-100 showed a moderate effect on activity up to a concentration of at least 10%, whereas incubation with the anionic surfactant SDS at 0.1% resulted in almost complete loss of activity. This was also the case for the chelating agent EDTA. The reducing agent p-mercaptoethanol also exhibited a moderate concentration-dependent effect on activity.

### Detergents

The activity of Amy_{I3C6} in three commercial detergents was determined using both a Tris-HCl buffer system and standard tap water (Table 3). Amy_{I3C6} was active in the two detergents Green Balance (solid) and Bio-tex (liquid), both of which are detergents with amylases added, although activity was somewhat lower than the buffer control. Interestingly, using tap water in the assays completely restored activity in the two detergents. No activity was observed in the detergent Neutral (solid), which only contains proteases.

TABLE 3
Effect of commercial detergents on Amy_{I3C6} activity. Assays were
run in Tris-HCl buffer or tap water adjusted to pH 8.6 with HCl and the
activity in buffer without detergents was set to 100%. Standard
deviations are shown in parentheses.

| | Relative activity | | | |
|---|---|---|---|---|
| Commercial detergents | Buffer | | Tap water | |
| None | 100% | (±11%) | 34% | (±2%) |
| Solid detergents | | | | |
| Green Balance (SuperGros A/S, | 31% | (±2%) | 116% | (±2%) |
| Denmark)* | | | | |
| Neutral General Purpose (Unilever | −6% | (±1%) | 2% | (±2%) |
| Denmark A/S) | | | | |
| Liquid detergent | | | | |
| Bio-tex White (Unilever Denmark | 43% | (±3%) | 97% | (±2%) |
| A/S)* | | | | |

*Detergents containing amylase. The results have been adjusted accordingly.

### Chromatographic Analysis of Hydrolysis Products

The hydrolysis products of Amy_{I3C6} acting on various polysaccharides and maltooligosaccharides of varying lengths (G2 to G7) were analyzed by thin-layer chromatography (TLC). Amy_{I3C6} was capable of hydrolyzing amylopectin, amylose and hydrolyzed starch to yield maltose (G2) and larger maltooligosaccharides, whereas no activity was observed on granular starch or glycogen. Maltoheptaose (G7) was hydrolyzed to G2-G4, maltopentaose (G5) to G2-G3 and weak activity was observed on maltotetraose (G4), which was hydrolyzed to G2. No activity was observed on G2 and G3. The results suggest that Amy_{I3C6} is an endo-acting enzyme, which prefers at least three sugar residues on one side of the cleavage site and at least two for cleavage.

### DISCUSSION

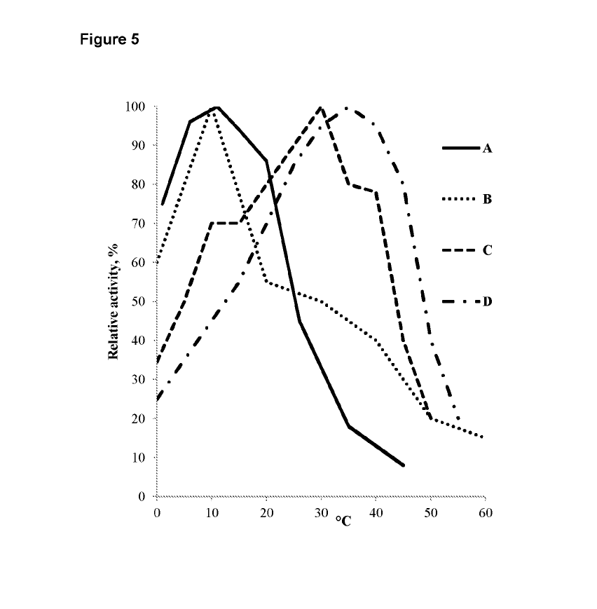

A phylogenetic analysis clustered Amy_{I3C6} with putative alpha-amylases of the class Clostridia with the two closest relatives being from Finegoldia magna (Goto et al. 2008) and Relcococcus kunzii (Collins et al. 1993). The previously characterized alpha-amylases from Clostridia are mainly thermophilic (Sivakumar et al. 2006; Ueki et al. 1991; Sai et al. 1991), although the alpha-amylase from the mesophilic Clostridium perfringens has an optimum at 30° C. and retains 70% of its activity at 15° C. (Shih and Labbe 1995). Not many cold-active alpha-amylases have been reported. A natural isolate from Svalbard related to Actinobacteria (Groudieva et al. 2004) and a soil isolate related to Bacillus (Mojallali et al. 2013) both showed alpha-amylase activity with an optimum at 37° C. and retained 20% and 13% of the activity at 0° C., respectively. An earthworm alpha-amylase showed 25% activity at 10° C. (Ueda et al. 2008), an alpha-amylase of Bacillus cereus showed activity over a broad temperature range with optimum at 50° C. and retained 50% of the activity at 10° C. (Mandavi et al. 2010), and the alpha-amylase from the psychrophilic soil bacterium Aeromonas veronii has an optimum at 10° C., and approximately 60% activity at 0° C. (Samie et al. 2012). The alpha-amylase from Pseudoalteromonas arctica G5230 (Lu et al. 2010) and from a strain related to Nocardiopsis (Zhang and Zeng 2008) retained 34.5% and ˜25% of the activity at 0° C., respectively. The optimum temperature of Amy_{I3C6} is at 10-15° C. and it retains more than 70% of its activity at 1° C. (FIG. 3), which, to the best of our knowledge, makes it the most psychrophilic alpha-amylase characterized so far in terms of relative activity at low temperature (FIG. 5). Another extremely cold-active alpha-amylase was isolated from Aeromonas veronii, but in contrast to Amy_{I3C6}, this has a low pH optimum (Samie et al. 2012).

Cold-adapted enzymes are characterized by their flexible structures, which allows for activity at low temperatures. This is partly achieved by decreasing the number of arginine and proline residues. The rigid proline residues are avoided in turns and loops leading to a lower overall abundance. Arginine contributes to stability in thermally adapted enzymes, since it is capable of forming more than one salt bridge and up to five hydrogen bonds. Consequently, the low abundances of proline and arginine residues and the arginine/(arginine+lysine) ratio can be used as an indication of cold-adaption (Feller and Gerday 1997). In all these measures, the alpha-amylase Amy_{I3C6} originating from the cold and alkaline ikaite columns of SW Greenland displays an even more pronounced cold-adaptation than the well characterized alpha-amylase from the psychrophilic P. haloplanctis (AHA), indicating that Amy_{I3C6} is a cold-adapted enzyme. Alignment of the amino acid sequence of Amy_{I3C6} shows that residues involved in catalytic activity and substrate binding are conserved compared to alpha-amylases from psychrophiles and mesophiles, as well as from plants and Archaea, indicating that the mode of action of Amy_{I3C6} is most likely similar to that of known amylases.

Cold-active amylases can be used in detergents to facilitate efficient washing at lower temperatures thus saving energy and reducing washing time. Since the pH of detergents is high, any added enzymes must be alkali tolerant (Gupta et al. 2002). The optimal pH for activity of Amy_{I3C6} was at approximately 8.6 and it retained more than 80% of its activity at pH 9 and was still active at pH 10. Stainzyme®, a commercially available alpha-amylase used for low temperature washing, has a broad temperature range of activity, but the activity decreases drastically below 20° C. At 10° C., the activity of Stainzyme® had decreased to 20% and almost no activity was observed at 1° C. Amy_{I3C6}, on the other hand, retained more than 70% of its activity at 1° C., clearly illustrating the psychrophilic properties of Amy_{I3C6} and indicating that it might serve as a starting point for a commercial, cold-active alpha-amylase for detergent formulations.

Cold-active amylases can also be applied in the food and feed industry. In the baking industry, alpha-amylases can be used to reduce the dough fermentation time, improve the properties of the dough and the crumb and the retention of aromas and moisture levels, as well as prevent stalling (Gerday et al. 2000; Cavicchioli et al. 2011). The use of cold-active amylases can be advantageous not only because of their general higher specific activity leading to reduced amounts required, but also because they can be easily inactivated, and inactivation is necessary to prevent prolonged activity resulting in undesired crumb structure (Gerday et al. 2000; Coronado et al. 2000). Amy_{I3C6} was heat-labile and easily inactivated at higher temperatures, suggesting that it could be successfully applied in the food and feed industry.

Production of cold-active enzymes in a mesophilic host like E. coli can be problematic due to thermal instability and improper folding. Amy_{I3C6} was produced in E. coli at 20° C. after biomass build-up at 37° C. Feller et al. (1998) found that 18° C. was the best compromise for production of the cold-active alpha-amylase from P. haloplanctis (AHA) in E. coli, and similar conditions could be expected for the optimal production of Amy_{I3C6} in E. coli.

The activity of Amy_{I3C6} was sensitive to various metal ions, especially Fe²⁺, Cu²⁺ and Zn²⁺ (Table 2). A similar effect of Fe²⁺ and Ce²⁺ was seen for the earthworm alpha-amylase (Ueda et al. 2008), and of Cu²⁺ for a thermostable alpha-amylase from Bacillus licheniformis NH1 (Hmidet et al. 2008). Surprisingly, Amy_{I3C6} also appeared to be inhibited by Ca²⁺, however this effect was not observed for CaCO₃. The chelating agent EDTA resulted in complete loss of activity (FIG. 4), indicating that a divalent metal ion is required for activity and normally Ca²⁺ is required for alpha-amylase activity (Machius et al. 1998). The anionic surfactant SDS resulted in complete loss of activity at low concentrations (0.1%), whereas moderate concentrations of the non-ionic surfactants Tween 20 and Triton X-100 (up to 10%) were tolerated by Amy_{I3C6} as was the reducing agent β-mercaptoethanol. In addition, the activity of Amy_{I3C6} was tested in the complex environment of three commercial detergents at concentrations simulating washing conditions. The loss of activity in the presence of the chelating agent EDTA would suggest that the activity of Amy_{I3C6} would be reduced in detergents containing chelating agents to minimize the effects of water hardness. A decrease in activity was observed when detergent was dissolved in buffer, but interestingly, full activity was restored when two of the detergents were dissolved in tap water, indicating that Amy_{I3C6} could be functional under washing conditions. Amy_{I3C6} showed full activity in both a solid (Green Balance) and a liquid (Bio-tex) detergent. Both of these detergents have amylases added and might therefore be optimized for amylase activity. The solid detergent where Amy_{I3C6} was inactive only contains proteases, possibly meaning a less favorable environment for amylases. The fact that Amy_{I3C6} had high activity in both liquid and solid detergents and was not stimulated by Ca²⁺ is similar to the properties of the thermostable alpha-amylase from Bacillus licheniformis NH1 (Hmidet et al. 2008).

Amy_{I3C6} was able to hydrolyze amylopectin, amylose and hydrolyzed starch down to maltose, which is similar to the alpha-amylase from Bacillus cereus (Mandavi et al. 2010), but in contrast to the alpha-amylase from Bacillus licheniformis NH1, which also released glucose (Hmidet et al. 2008). No activity was observed on granular starch and glycogen. This is not unexpected given the lack of a starch binding domain in Amy_{I3C6}, which is normally required for hydrolysis of granular starch (Christiansen et al. 2009). The lack of activity on glycogen could be due to the highly branched nature of this substrate.

A recombinantly produced cold-adapted alpha-amylase Amy_{I3C6} identified in a metagenomic library from the cold and alkaline ikaite columns of SW Greenland was found to be an extremely cold-active alpha-amylase, retaining more than 70% of its activity at 1° C. The enzyme displayed optimal activity at 10-15° C. and a pH of 8-9. Sequence analysis clustered Amy_{I3C6} with alpha-amylases related to Clostridia and the enzyme showed strong psychrophilic adaptations. Amy_{I3C6} displayed full activity in both a solid and liquid detergent, which together with the temperature and pH profiles suggests that this alpha-amylase could be a useful starting point for engineering of a cold-active alpha-amylase for use in the detergent industry.

### REFERENCES

Andersen, D. T., Sumner, D. Y., Hawes, I., Webster-Brown, J., and McKay, C. P. 2011. Discovery of large conical stromatolites in Lake Untersee, Antarctica. Geobiology 9: 280-293.

Anthon, G. E. and Barrett, D. M. 2002. Determination of reducing sugars with 3-methyl-2-benzothiazolinonehydrazone. Anal. Biochem. 305: 287-289.

Asgher, M., Asad, M. J., Rahman, S. U., and Legge, R. L. 2007. A thermostable alpha-amylase from a moderately thermophilic Bacillus subtilis strain for starch processing. Journal of Food Engineering 79: 950-955.

Bisgaard-Frantzen, H., Svendsen, A., Norman, B., Pedersen, S., Kjaerulff, S., Outtrup, H., and Borchert, T. V. 1999. Development of Industrially Important alpha-Amylases. Journal of Applied Glycoscience 46: 199-206.

Buchardt, B., Seaman, P., Stockmann, G., Vous, M., Wilken, U., Duwel, L., Kristiansen, A., Jenner, C., and Whiticar, M. J. 1997. Submarine columns of ikaite tufa. Nature 390: 129-130.

Cavicchioli, R., Charlton, T., Ertan, H., Mohd, O. S., Siddiqui, K. S., and Williams, T. J. 2011. Biotechnological uses of enzymes from psychrophiles. Microb. Biotechnol. 4: 449-460.

Christiansen, C., Abou, H. M., Janecek, S., Vikso-Nielsen, A., Blennow, A., and Svensson, B. 2009. The carbohydrate-binding module family 20—diversity, structure, and function. FEBS J 276: 5006-5029.

Cipolla, A., D'Amico, S., Barumandzadeh, R., Matagne, A., and Feller, G. 2011. Stepwise adaptations to low temperature as revealed by multiple mutants of psychrophilic alpha-amylase from Antarctic Bacterium. J. Biol. Chem. 286: 38348-38355.

Collins, M. D., Facklam, R. R., Rodrigues, U. M., and Ruoff, K. L. 1993. Phylogenetic analysis of some Aerococcus-like organisms from clinical sources: description of Helcococcus kunzii gen. nov., sp. nov. Int. J Syst. Bacteriol. 43: 425-429.

Coronado, M., Vargas, C., Hofemeister, J., Ventosa, A., and Nieto, J. J. 2000. Production and biochemical characterization of an alpha-amylase from the moderate halophile Halomonas meridiana. PENS Microbiol. Lett. 183: 67-71.

Feller, G. and Gerday, C. 1997. Psychrophilic enzymes: molecular basis of cold adaptation. Cell Mol. Life Sci. 53: 830-841.

Feller, G., Le, B. O., and Gerday, C. 1998. Expression of psychrophilic genes in mesophilic hosts: assessment of the folding state of a recombinant alpha-amylase. Appl. Environ. Microbiol. 64: 1163-1165.

Gerday, C., Aittaleb, M., Bentahir, M., Chessa, J. P., Claverie, P., Collins, T., D'Amico, S., Dumont, J., Garsoux, G., Georlette, D., Hoyoux, A., Lonhienne, T., Meuwis, M. A., and Feller, G. 2000. Cold-adapted enzymes: from fundamentals to biotechnology. Trends Biotechnol. 18: 103-107.

Goto, T., Yamashita, A., Hirakawa, H., Matsutani, M., Todo, K., Ohshima, K., Toh, H., Miyamoto, K., Kuhara, S., Hattori, M., Shimizu, T., and Akimoto, S. 2008. Complete genome sequence of Finegoldia magna, an anaerobic opportunistic pathogen. DNA Res. 15: 39-47.

Groudieva, T., Kambourova, M., Yusef, H., Royter, M., Grote, R., Trinks, H., and Antranikian, G. 2004. Diversity and cold-active hydrolytic enzymes of culturable bacteria associated with Arctic sea ice, Spitzbergen. Extremophiles. 8: 475-488.

Gupta, R., Beg, Q. K., and Lorenz, P. 2002. Bacterial alkaline proteases: molecular approaches and industrial applications. Appl. Microbiol. Biotechnol. 59: 15-32.

Gupta, R., Gigras, P., Mohapatra, H., Goswami, V. K., and Chauhan, B. 2003. Microbial alpha-amylases: a biotechnological perspective. Process Biochemistry 38: 1599-1616.

Hmidet, N., Bayoudh, A., Berrin, J. G., Kanoun, S., Juge, N., and Nasri, M. 2008. Purification and biochemical characterization of a novel alpha-amylase from Bacillus licheniformis NH1—Cloning, nucleotide sequence and expression of amyN gene in Escherichia coli. Process Biochemistry 43: 499-510.

Jungblut, A. D., Wood, S. A., Hawes, I., Webster-Brown, J., and Harris, C. 2012. The Pyramid Trough Wetland: environmental and biological diversity in a newly created Antarctic protected area. FEMS Microbiol. Ecol. 82: 356-366.

Kirk, O., Borchert, T. V., and Fuglsang, C.C. 2002. Industrial enzyme applications. Curr. Opin. Biotechnol. 13: 345-351.

Linden, A. and Wilmanns, M. 2004. Adaptation of class-13 alpha-amylases to diverse living conditions. Chembiochem. 5: 231-239.

Lu, M., Wang, S., Fang, Y., Li, H., Liu, S., and Liu, H. 2010. Cloning, expression, purification, and characterization of cold-adapted alpha-amylase from Pseudoalteromonas arctica G5230. Protein J 29: 591-597.

Machius, M., Declerck, N., Huber, R., and Wiegand, G. 1998. Activation of Bacillus licheniformis alpha-amylase through a disorder-->order transition of the substrate-binding site mediated by a calcium-sodium-calcium metal triad. Structure. 6: 281-292.

Mandavi, A., Sajedi, R. H., Rassa, M., and Jafarian, V. 2010. Characterization of an a-Amylase with Broad Temperature Activity from an Acid-Neutralizing Bacillus cereus Strain. Iran J Biotech 8.

Mojallali, L., Shahbani, Z. H., Rajaei, S., Akbari, N. K., and Haghbeen, K. 2013. A novel approximately 34-kDa alpha-amylase from psychrotroph Exiguobacterium sp. SH3: Production, purification, and characterization. Biotechnol. Appl. Biochem.

Pandey, A., Nigam, P., Soccol, C. R., Soccol, V. T., Singh, D., and Mohan, R. 2000. Advances in microbial amylases. Biotechnology and Applied Biochemistry 31: 135-152.

Petersen, T. N., Brunak, S., von, H. G., and Nielsen, H. 2011. SignalP 4.0: discriminating signal peptides from transmembrane regions. Nat. Methods 8: 785-786.

Sai, R. M., Rao, C. V., and Seenayya, G. 1991. Characteristics of Clostridium thermocellum strain SS8: A broad saccharolytic thermophile. World J Microbiol. Biotechnol. 7: 272-275.

Samie, N., Noghabi, K. A., Gharegozloo, Z., Zahiri, H. S., Ahmadian, G., Sharafi, H., Behrozi, R., and Vali, H. 2012. Psychrophilic alpha-amylase from Aeromonas veronii NS07 isolated from farm soils. Process Biochemistry 47: 1381-1387.

Schmidt, M., Prieme, A., and Stougaard, P. 2006. Bacterial diversity in permanently cold and alkaline ikaite columns from Greenland. Extremophiles. 10: 551-562.

Shih, N. J. and Labbe, R. G. 1995. Purification and characterization of an extracellular alpha-amylase from Clostridium perfringens type A. Appl. Environ. Microbiol. 61: 1776-1779.

Sivakumar, N., Li, N., Tang, J. W., Patel, B. K., and Swaminathan, K. 2006. Crystal structure of AmyA lacks acidic surface and provide insights into protein stability at poly-extreme condition. FEBS Lett. 580: 2646-2652.

Stougaard, P., Jorgensen, F., Johnsen, M. G., and Hansen, O. C. 2002. Microbial diversity in ikaite tufa columns: an alkaline, cold ecological niche in Greenland. Environ. Microbiol. 4: 487-493.

Tamura, K., Stecher, C., Peterson, D., Filipski, A., and Kumar, S. 2013. MEGA6: Molecular Evolutionary Genetics Analysis version 6.0. Mol. Biol. Evol. 30: 2725-2729.

Ueda, M., Asano, T., Nakazawa, M., Miyatake, K., and Inouye, K. 2008. Purification and characterization of novel raw-starch-digesting and cold-adapted alpha-amylases from Eisenia foetida. Comp Biochem. Physiol B Biochem. Mol. Biol. 150: 125-130.

Ueki, A., Hirono, T., Sato, E., Mitani, A., and Ueki, K. 1991. Ethanol and amylase production by a newly isolated Clostridium sp. World J Microbiol. Biotechnol. 7: 385-393.

van der Maarel, M. J., van, d., V, Uitdehaag, J. C., Leemhuis, H., and Dijkhuizen, L. 2002. Properties and applications of starch-converting enzymes of the alpha-amylase family. J. Biotechnol. 94: 137-155.

Vester, J. K., Glaring, M. A., Lopez, A. G., and Stougaard, P. 2014. Discovery of novel industrial enzymes from a cold and alkaline environment by a combination of functional metagenomics and culturing. Submitted to Microb. Cell Fact.

Vester, J. K., Lylloff, J. E., Claring, M. A., and Stougaard, P. 2013. Microbial Diversity and Enzymes in Ikaite Columns; A Cold and Alkaline Environment in Greenland. In Polyextremophiles—Life Under Multiple Forms of Stress. Springer.

Wang, N., Zhang, Y., Wang, Q., Liu, J., Wang, H., Xue, Y., and Ma, Y. 2006. Gene cloning and characterization of a novel alpha-amylase from alkaliphilic Alkalimonas amylolytica. Biotechnol. J 1: 1258-1265.

Zhang, J. W. and Zeng, R. Y. 2008. Purification and characterization of a cold-adapted alpha-amylase produced by Nocardiopsis sp. 7326 isolated from Prydz Bay, Antarctic. Mar. Biotechnol. (NY) 10: 75-82.

1. A purified cold-active alpha-amylase having the amino acid sequence as defined in SEQ ID NO 1 or one having at least 80% homology to the amino acid sequence as defined in SEQ ID NO 1, the amino acid sequence being selected so that the enzyme has a stable enzymatic activity at temperatures less than 10° C.

2. An alpha-amylase according to claim 1, wherein the amino acid sequence has at least 90%, preferably 95%, homology to the amino acid sequence as defined in SEQ ID NO 1.

3. An isolated DNA sequence comprising a gene which encodes the alpha-amylase according to claim 1.

4. An isolated DNA sequence, which
a) encodes a protein with an amino acid sequence as given in SEQ ID NO. 1, or
b) hybridises under stringent conditions to the DNA sequence of a), or
c) is degenerative of the sequence of a) or b).

5. A DNA sequence according to claim 4, wherein the sequence is as given in SEQ ID NO. 2.

6. A recombinant vector comprising a DNA sequence of claim 3.

7. A vector of claim 6, wherein said vector is an expression vector.

8. A host cell transformed with a vector of claim 6.

9. A cell according to claim 8, wherein the cell is selected from the group consisting of Escherichia, Bacillus, Bifidobacterium, Lactococcus, Lactobacillus, Streptomyces, Leuconostoc, Streptomyces, Saccharomyces, Kluyveromyces, Candida, Torula, Torulopsis, Pichia pastoris, and Aspergillus.

10. A process for producing an enzyme of claim 1, comprising culturing a cell in a suitable culture medium under conditions permitting expression of said enzyme, and recovering the resulting enzyme from the culture.
